(19) United States
(12) Patent Application Publication
(10) Pub. No.: US 2012/0109080 A1
(43) Pub. Date: May 3, 2012

(54) PUNCTURE RESISTANT COMPOSITE MATERIALS

(75) Inventors: Nicholas J. Manesis, Summerland, CA (US); Kaustubh S. Chitre, Goleta, CA (US); Dustin Leslie, Santa Barbara, CA (US); Nikhil S. Trilokekar, Goleta, CA (US)

(73) Assignee: ALLERGAN, INC., Irvine, CA (US)

(21) Appl. No.: 13/286,767

(22) Filed: Nov. 1, 2011

Related U.S. Application Data
(60) Provisional application No. 61/409,449, filed on Nov. 2, 2010.

(51) Int. Cl. A61M 5/00; B23P 17/04

(52) U.S. Cl. 604/288.01; 29/428

(57) ABSTRACT

A needle guard assembly includes a first composite guard and a second composite guard, each including an arrangement of puncture resistant members on a flexible substrate. The first composite guard and the second composite guard are positioned such that the arrangement of puncture resistant members of the second composite guard are misaligned with the arrangement of puncture resistant members of the first composite guard. The assembly further includes an intermediate layer disposed between and connecting the first composite guard with the second composite guard.

### RELATED APPLICATIONS

This application claims the benefit of, and priority to, U.S. Provisional Patent Application No. 61/409,449 filed on Nov. 2, 2010, and U.S. patent application Ser. No. 13/178,392 filed on Jul. 7, 2011, which is a continuation-in-part of U.S. patent application Ser. No. 13/105,715 filed on May 11, 2011, which is a continuation-in-part of U.S. patent application Ser. No. 13/021,523 filed on Feb. 4, 2011 which claims the benefit of U.S. Provisional Patent Application No. 61/409,440 filed Nov. 2, 2010, and the benefit of U.S. Provisional Patent Application No. 61/301,910 filed Feb. 5, 2010, the entire disclosure of each of these applications being incorporated herein by this reference.

### FIELD OF THE INVENTION

The present invention generally relates to puncture resistant materials and more specifically relates to puncture resistant materials, including implantable puncture resistant materials.

### BACKGROUND

Puncture resistant or puncture proof materials and fabrics for protection of the human body are well known. Puncture resistant materials are used for form protective armor, gloves, helmets, boots, and shields.

Protective materials that are of a solid composition limit freedom of movement. Attempts have been made to develop materials that are supple, bendable, twistable or otherwise flexible which also provide adequate resistance to penetration and/or cutting forces.

There is a need for more versatile protective resistant materials, for example, but not limited to, puncture resistant materials safe for implantation in a human body.

### SUMMARY OF THE INVENTION

Accordingly, the present invention provides assemblies, for example, puncture resistant assemblies and methods of making the same. In some aspects of the invention, puncture resistant assemblies are provided which are useful as components of surgical implants, for example, but not limited to, needle guards as components of inflatable implants that are accessed with a needle and syringe. Such implants for which the present materials are useful include inflatable tissue expanders. Other implants that can benefit from the present invention include fluid access ports which include a fluid reservoir and needle penetratable septum. In these and other implantable devices, puncture resistant or puncture proof assemblies of the invention can be highly beneficial, for example, as a means for preventing a needle tip from penetrating other areas of the device that are not intended to be punctured. Other beneficial uses for the present assemblies will become more apparent upon reading the present specification, and are considered to be included within the scope of the invention.

In one aspect of the invention, puncture resistant assemblies are provided which are flexible and/or formable into desired configurations. In some embodiments, puncture resistant assemblies are provided which are both flexible and resilient. Some of the present assemblies have the characteristic of shape memory, such that after being rolled or folded, they can resume an original shape or configuration. This aspect of the invention is particularly, but certainly not exclusively, useful for application in a surgical environment, in which the assembly may be in the form of a puncture proof material is rolled or folded into a narrow configuration, thereby enabling insertion thereof through a relatively small incision. Advantageously, some of the assemblies of the invention are structured to be able to automatically resume an original, pre-deformed shape, for example, automatically, once the material is at the desired implantation site.

In one embodiment of the invention, a puncture resistant assembly is provided which generally comprises a first composite guard, a second composite guard, and an intermediate layer securing the first and second composite guards together and/or containing the first and second composite guards.

Each of the first and second composite guards generally comprises an arrangement of puncture resistant elements or members, and a flexible substrate on which the members are secured and positioned, generally in a spaced-apart relationship.

The members may be in the form of domes or plates. The members have a hardness effective to resist penetration, puncture or breakage upon forceful contact with a sharp surface, for example, a tip of a needle, an edge of a cutting implement such as a scalpel or knife, or the like. The members may be made of any suitable material, such as a hard moldable substance, for example, a high durometer elastomer, polymer or rubber. Other suitable materials include metals, ceramics, and alloys thereof.

The flexible substrate on which the members are disposed may comprise a fabric, mesh, film, elastomer, or other material.

Notably, the first composite guard and the second composite guard are disposed with respect to one another such that the arrangement of members of the first composite guard is offset or misaligned with respect to the arrangement of members of the second composite guard. In some embodiments, a third composite guard is provided. The third composite guard may be positioned with respect to the first and second composite guards such that the members of the third composite guard are misaligned with the members of at least one of the first and second composite guards.

Advantageously, the misaligned or overlapping members of the adjacent composite guards provide a puncture resistant, or puncture proof, area while not significantly sacrificing flexibility of the assembly as a whole. That is, the composite guards may be arranged such that there are no significant gaps between individual puncture resistant members. It can be appreciated that depending upon the use of the final assembly, there may be some gaps between members so long as the gaps are sufficiently narrow to resist or prevent penetration by the type of instrument that the assembly is intended to be protected against puncture from.

In any event, in some embodiments of the invention, the puncture resistant members of the composite guards may provide a area of protection that substantially entirely covers a first side of the needle guard assembly.

The assembly may further comprise a intermediate layer, for example, an elastomer, securing together the first and second composite guards such that the members maintain their offset relationship. The intermediate layer may be located between adjacent composite guards and may be bonded thereto. In one embodiment, the intermediate layer seals the flexible composite members together and encapsulates the composite guards. For example, the intermediate layer may be an fluid tight barrier containing the two or more layered composite guards. In some embodiments, the intermediate layer exhibits a springiness and resiliency or provides a shape memory characteristic to the assembly.

In another aspect of the invention, a method of making a needle guard assembly is provided wherein the method generally comprises the steps of providing first and second composite guards where each composite guard includes a layer of puncture resistant members secured to a flexible substrate and bonding the first composite guard with the second composite guard in such that the members of the first composite guard are misaligned with the members of the second composite guard. In some embodiments, the method includes the step of bonding a third composite guard to the second composite guard such that the members of the third composite guard are misaligned with the members of at least one of the first composite guard and the second composite guard.

In some embodiments, the method may comprise the step of providing an intermediate layer between the composite guards. In some embodiments, the method may comprise the step of encasing or encapsulating the composite guards in a fluid tight seal.

In another aspect of the invention, an inflatable implantable device is provided which includes a puncture resistant assembly as described herein.

Each and every feature described herein, and each and every combination of two or more of such features, is included within the scope of the present invention provided that the features included in such a combination are not mutually inconsistent.

### BRIEF DESCRIPTION OF THE DRAWINGS

The present invention may be more clearly understood and certain aspects and advantages thereof better appreciated with reference to the following Detailed Description when considered with the accompanying Drawings of which:

FIG. 1 is cross-sectional view of a puncture resistant assembly in accordance with an embodiment of the invention, the puncture resistant assembly being shown as a component of an inflatable breast prosthesis;

FIG. 2 is an exploded view of the prosthesis shown in FIG. 1 in order to illustrate certain components of the puncture resistant assembly;

FIG. 3 is a top view of a composite guard which is a component of the puncture resistant assembly shown in FIG. 1;

FIG. 4 is a magnified view of a portion of the composite guard encompassed by line 4 of FIG. 3;

FIG. 5 is a cross-sectional view of the composite guard taken along line 5-5 of FIG. 4;

FIG. 6 is a cross-sectional view, similar to the view shown in FIG. 5, of an alternative composite guard in accordance with certain aspects of the invention;

FIG. 6a is a cross-sectional view, similar to the view shown in FIG. 5, of yet another composite guard in accordance with certain aspects of the invention;

FIGS. 7-9 illustrate steps useful in making some of the puncture resistant assemblies of the present invention.

### DETAILED DESCRIPTION

The present invention provides puncture resistant assemblies. The assemblies are useful, for example, in the prevention of needle puncture in any number of devices which would beneficially include such a puncture resistant assembly as described herein.

For example, the assemblies of the invention may be useful components of an implantable device, such as an inflatable prosthesis, a fluid access port, or any number of devices that is implanted in a body and which is accessed by a needle but which requires a portion to function as a needle stop. The materials can be used, for example, as a needle stop of an inflatable tissue expander.

Other uses for the assemblies of the invention include, but are not limited to, personal items and effects such as gloves, garments and equipment. Such items include garments for use in the medical industry, such as gloves for surgical use. Other uses include garments and equipment for sports and outdoors enthusiasts, for example, people engaged in fishing, hunting, and similar activities in which a puncture resistant material would be beneficial. Other uses include uniforms for protecting a wearer from industrial injuries in factories, on construction sites, in solid waste handling facilities, and in other such hazardous environments.

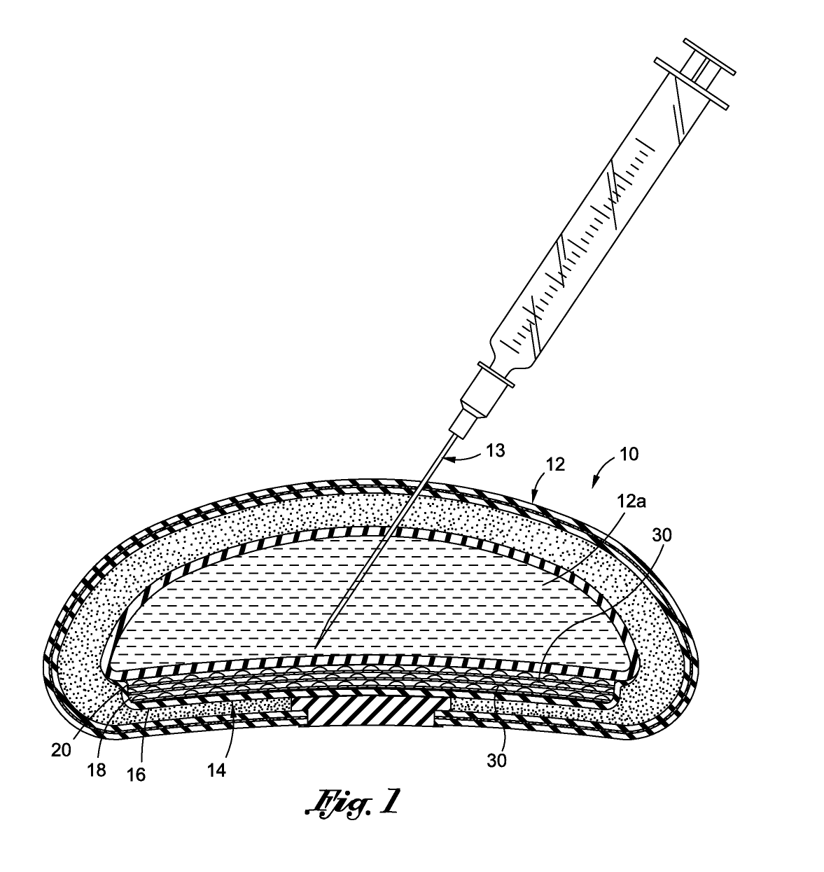

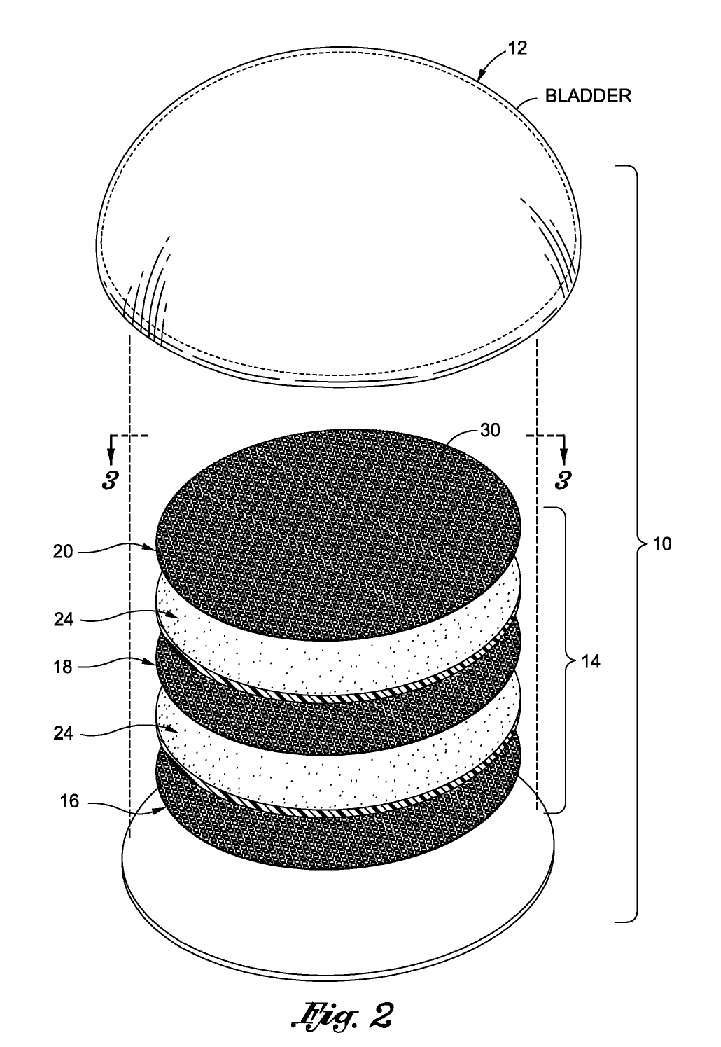

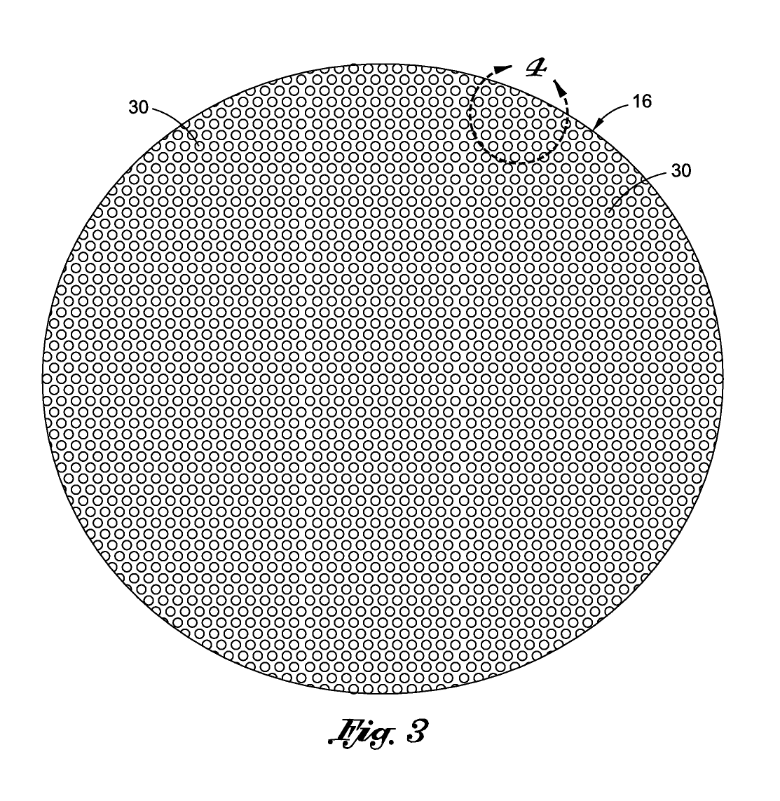

Turning now to FIGS. 1 and 2, an implantable, inflatable device 10 including a puncturable portion 12, and a puncture resistant assembly 14 in accordance with an embodiment of the invention, is shown in cross sectional view and exploded view, respectively, in order to illustrate one useful application of the assemblies of the present invention.

Device 10 is expanded or inflated by insertion of a needle 13 (FIG. 1) through bladder which form a fillable portion 12 and introduction of fluid into cavity 12a. Portion may include an access port (not shown) with a needle penetratable septum, or may be made partially or entirely of a puncturable, but self sealing material. Suitable self sealing materials are described in U.S. Provisional Patent Application No. 61/301,910, filed on Feb. 10, 2010 and in U.S. patent application Ser. No. 12/543,795, filed on Aug. 19, 2009, the entire specifications of which are incorporated herein by this reference. In order to prevent the needle 13 from undesirably penetrating through the device 10, the device can be equipped with assembly 14.

Referring now to FIG. 2, the assembly 14 generally comprises a first composite guard 16 and a second composite guard 18. In the shown embodiment, the assembly further includes a third composite guard 20. In other embodiments, only two composite guards or more than three composite guards are provided. An intermediate layer 24 is provided between adjacent guards, for example, between guard 16 and guard 18, and likewise between guard 18 and guard 20.

Each of composite guards 16, 18, 20 includes a plurality of, for example, an arrangement, array, or pattern of, puncture resistant members 30, and a flexible substrate 32 having a first side on which the puncture resistant members 30 are disposed in a generally spaced apart fashion.

As can be perhaps best appreciated from FIG. 1 (and FIG. 9), the first composite guard 16 and the second composite guard 18 are positioned such that the arrangement of puncture resistant members 30 of the second composite guard 18 are misaligned with the arrangement of puncture resistant members 30 of the first composite guard 16. Similarly, the second composite guard 18 and the third composite guard 20 may be positioned such that the arrangement of puncture resistant members of the third composite guard 20 are misaligned with the arrangement of puncture resistant members of at least one of the first composite guard 16 and the second composite guard 18. Thus, accordingly, the composite guards 16, 18, 20 are arranged relative to one another such that there are no straight line open spaces, or substantial gaps, between members 30 to allow a needle or sharp implement to penetrate entirely through the assembly 14. Yet, advantageously, the assembly 14 as a whole may be quite flexible in that the substrate 32 on which the spaced apart 30 members are disposed is supple, flexible and/or bendable.

Turning specifically to FIG. 2, the intermediate layer may comprise a flexible, connecting material which is effective to couple or bond the first composite guard 16 with the second composite guard 18, and the second composite guard 18 with the third composite guard 20. As shown in FIG. 2, the intermediate layer 24 is positioned between the arrangement of puncture resistant members 30 of the first layer 16 and the flexible substrate 32 of the second layer 18, and another intermediate layer 24 is positioned between the arrangement of puncture resistant members 30 of the second layer 18 and the flexible substrate 32 of the third layer 20.

The composite guards 16, 18, 20 may be identical to one another, and for the sake of simplicity, only the first composite guard 16 will now be described, with the understanding that, in the shown embodiment, what is described for the first composite guard 16 is also applicable to second composite guard 18 and third composite guard 20.

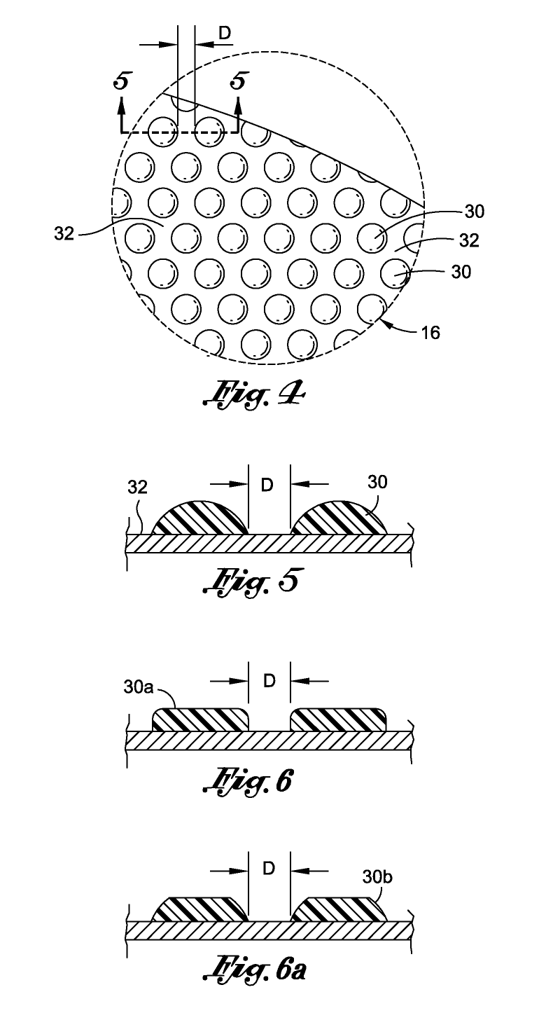

The members 30 may be any suitable shape. In FIG. 5, the members 30 are somewhat dome shaped with rounded surfaces. In other embodiments, members 30a may be planar as illustrated in FIG. 6. Alternatively still, the members 30b may include both rounded surface and planar or flat surfaces, such as the members 30b which are dome shaped with a flat upper surface, as illustrated in FIG. 6a.

The members 30 have a thickness of between about 0.1 mm and about 1.0 mm, for example, a thickness of between about 0.2 mm and about 0.5 mm for example, between about 0.1 mm and about 1.0 mm. The members 30 have a spacing D of between about 0.2 mm and about 0.5 mm. The members 30 have a diameter of between about 0.5 mm and about 2.0 mm, for example, a diameter of about 1.5 mm.

In some embodiments, the guard 16 includes between about 50 and about 1000 members per square inch (psi), for example, about 400 psi.

In a specific embodiment, the guard 16 include about 400 members psi, each having a diameter of about 1.5 mm and each being spaced apart about 0.2 mm.

The members 30 (and 30a and 30b) are made of a suitable puncture resistant material, such as an epoxy, polymer, rubber, cermamic or metal, or suitable combination or alloy thereof. For some applications, suitable materials include polyethylene (PE), polypropylene (PP), polyurethane (PU), polyethylene terephthalate (PET), piolycarbonate (PC), Polyisoprene (PI), thermoplastic urethanes and thermoplastic polyurethanes (TPU), high durometer silicones, acrylonitrile butadiene styrene (ABS) etc. In some embodiments, the members are made of material selected from acetal, nylon, and polycarbonate. In some embodiments, the members 330 are made of a metal, for example, stainless steel, aluminum, titanium, or other metal.

The flexible substrate 32 may comprise a mesh, film, fabric, elastomer, or other suitable material.

The intermediate layer 24 may be a polymer, for example, an elastomeric polymer, for example, a silicone elastomer, for example, a low durometer silicone rubber.

In some embodiments, the assembly 14 has a resiliency or a shape memory such that it will restore from a folded or rolled configuration to an original, different configuration. The original configuration may be a generally flat or planar configuration. This may be provided by using a suitable intermediate layer material, such as a silicone elastomer that has a shape memory characteristic.

Assembly of the guard assembly 14 may be accomplished as follows and as shown in FIGS. 7-9.

Turning now to FIG. 7, guard 16 generally comprising members 30 and substrate 32, is made by any suitable method, including stencil printing, for example, using equipment and processes used in surface mount technology/PCB fabrication. Other processes that can be used to make the guard 16 include micro-dot dispensing and printing, laser etching. Other suitable methods will be known to those of skill in the art.

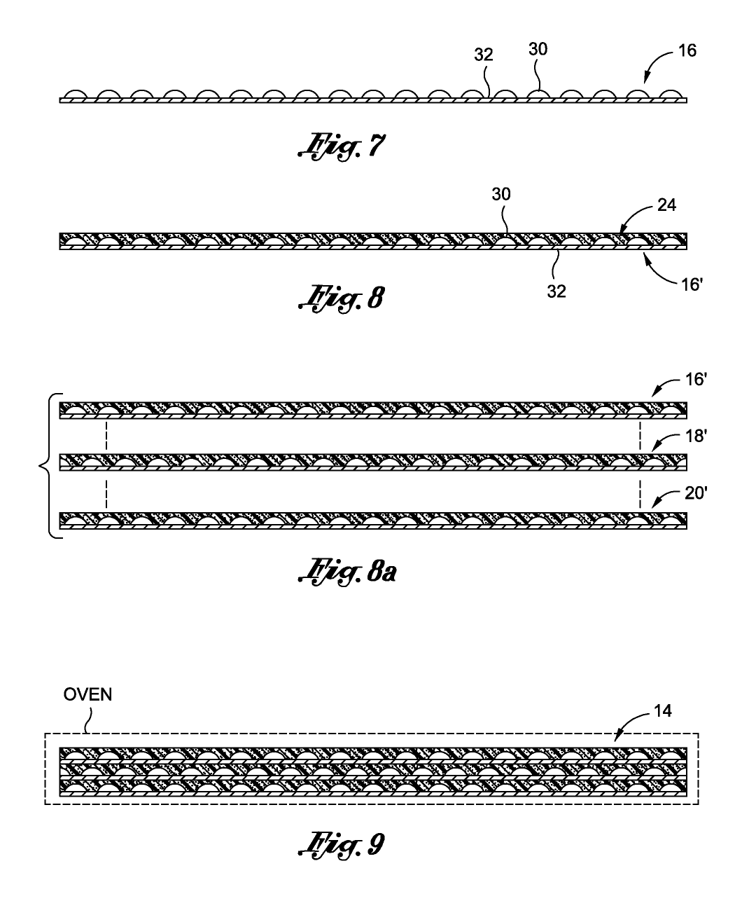

Turning to FIG. 8, intermediate layer 24 may be formed as follows. A suitable material, for example, a sheet of uncured silicone, is placed on one side of the guard 16, for example, on the side having members 30 and substrate 32. The sheet is then subjected to curing conditions to cause the sheet to adhere to the members 30, forming intermediate layer 24 thereon. In the presently described example embodiment, this step is done three times, with three separate guards 16, 18, 20, to form the components 16′, 18′ and 20′ of assembly 14. (See FIG. 8a).

The assembly 14 is then placed in an oven or otherwise subjected to further curing conditions to seal the assembly components together such as shown in FIG. 9.

While this invention has been described with respect to various specific examples and embodiments, it is to be understood that the invention is not limited thereto and that it can be variously practiced within the scope of the invention.

What is claimed is:

1. A needle guard assembly comprising:
a first composite guard and a second composite guard, each composite guard including an arrangement of puncture resistant members and a flexible substrate having a first side on which the puncture resistant members are disposed in a spaced apart fashion,
the first composite guard and the second composite guard being positioned such that the arrangement of puncture resistant members of the second composite guard are misaligned with the arrangement of puncture resistant members of the first composite guard; and
an intermediate layer disposed between and connecting the first composite guard with the second composite guard.

2. The assembly of claim 1 wherein the intermediate layer is positioned between the arrangement of puncture resistant members of the first layer and the flexible substrate of the second layer.

3. The assembly of claim 1 further comprising a third composite guard wherein the arrangement of puncture resistant members of the third composite guard are misaligned with the arrangement of puncture resistant members of the second composite guard.

4. The assembly of claim 1 wherein the puncture resistant members are dome shaped.

5. The assembly of claim 1 wherein the puncture resistant members include a flat surface area.

6. The assembly of claim 1 wherein the puncture resistant members are generally dome shaped with a flat surface area.

7. The assembly of claim 1 wherein the puncture resistant members are made of a material selected from acetal, nylon, and polycarbonate.

8. The assembly of claim 1 wherein the puncture resistant members are made of metal.

9. The assembly of claim 1 wherein the puncture resistant members are made of a material selected from stainless steel, aluminum, and titanium.

10. The assembly of claim 1 wherein the intermediate layer is an elastomer.

11. The assembly of claim 1 wherein the intermediate layer is silicone.

12. The assembly of claim 1 wherein the puncture resistant members are made of epoxy.

13. The assembly of claim 1 wherein the flexible substrate is a biocompatible film.

14. The assembly of claim 1 wherein the puncture resistant members have a thickness of between about 0.1 mm and about 1.0 mm.

15. The assembly of claim 1 wherein the puncture resistant members have a thickness of between about 0.2 mm and about 0.5 mm.

16. The assembly of claim 1 wherein the puncture resistant members have a spacing of between about 0.1 mm and about 1.0 mm

17. The assembly of claim 1 wherein the puncture resistant members have a spacing of between about 0.2 mm and about 0.5 mm.

18. The assembly of claim 1 wherein the puncture resistant members have a diameter of between about 0.5 mm and about 2.0 mm.

19. The assembly of claim 1 wherein the puncture resistant members have a diameter of about 1.5 mm.

20. A method of making a needle guard assembly, comprising the steps of:
providing a first composite guard and second composite guard, each composite guard including an arrangement of puncture resistant members and a flexible substrate having a first side on which the puncture resistant members are disposed in a spaced apart fashion; and
securing the first composite guard with the second composite guard such that the second arrangement of plates is misaligned with the first arrangement of plates in order to maintain no gaps therebetween.
